(19) United States
(12) Patent Application Publication
(10) Pub. No.: US 2025/0082395 A1
(43) Pub. Date: Mar. 13, 2025

(54) A MULTI-ELECTRODE PULSED FIELD ABLATION CATHETER FOR CREATION OF SPOT LESIONS

(71) Applicant: CathRx Ltd, Homebush Bay, NSW (AU)

(72) Inventors: Daniel Meckes, Springwood, NSW (AU); Milanjot Singh Assi, Kellyville Ridge, NSW (AU); Ian Kim Seng Fong, Lane Cove, NSW (AU); Pengjin Pan, Sydney, NSW (AU)

(21) Appl. No.: 18/292,635

(22) PCT Filed: Jun. 6, 2022
(86) PCT No.: PCT/IB2022/055222

§ 371 Date: Jan. 26, 2024

Related U.S. Application Data
(60) Provisional application No. 63/236,746, filed on Aug. 25, 2021.

(51) Int. Cl. A61B 18/14; A61B 18/00; A61B 18/16

(52) U.S. Cl. CPC A61B 18/1492; A61B 2018/00351; A61B 2018/00577; A61B 2018/165

(57) ABSTRACT

A mini-loop cardiac ablation catheter having a shaft having a proximal end, a distal end, and at least one loop extending from the distal end. A handle is coupled to the proximal end of the shaft, the handle having a steering mechanism. A plurality of electrodes are positioned on the loop electrically coupled to at least one electrical connector in the handle. the at least one electrical connector being configured to electrically coupled with an electrical ablation energy source to power the plurality of electrodes.

### CROSS-REFERENCE TO RELATED APPLICATIONS

This application is a national phase entry under 35 U.S.C. § 371 of International Patent Application PCT/IB2022/055222, filed Jun. 6, 2022, designating the United States of America and published as International Patent Publication WO 2023/026106 A1 on Mar. 2, 2023, which claims the benefit under Article 8 of the Patent Cooperation Treaty of U.S. Provisional Patent Application Ser. No. 63/236,746, filed Aug. 25, 2021.

### TECHNICAL FIELD

The present disclosure relates to a steerable cardiac catheter and, more particularly, to an intracardiac ablation catheter, systems and improvements thereto.

### BACKGROUND

In the conduction of Cox-Maze type procedures, an ablation catheter is used to ablate heart tissue to attempt to clear heart arrhythmias. Generally, a dot or spot ablation is made and this is repeated by re-positioning a tip and the ablation electrode of an ablation catheter. This is an extremely time-consuming process. In addition, dot or spot ablation may leave gaps in the lesions, which may again require re-positioning and repeating the procedure. If a clinician could form longer lesions, fewer manipulations would be required. This would reduce the time to conduct the procedure, which would be beneficial for all concerned. Longer electrodes have been considered for radiofrequency ablation, but coagulum tends to form on the electrodes. In addition, the energy field from long electrodes is not always uniform and this may cause discontinuities in the lesion. Furthermore, the temperature of the ablation electrodes as well as the tissue being treated needs to be carefully maintained to ensure that it does not result in excessive ablation of the tissue.

Catheter ablation of atrial fibrillation (AF) using thermal energies such as radiofrequency or cryotherapy is associated with indiscriminate tissue destruction. Radiofrequency (RF) is a widely accepted energy source for myocardial ablation but may result in discontinuous lesions and nontargeted tissue injury. Further, applying RF energy to atrial wall tissue can damage the esophagus or nerves that are in the region close to the heart. RF ablation procedures may potentially require an extended period of treatment time to correct the arrythmia.

During pulsed field ablation (PFA), sub-second electric fields create microscopic pores in cell membranes-a process called electroporation. Irreversible electroporation can be used as a nonthermal energy source to ablate tissue. Cardiac catheter ablation by irreversible electroporation may be a safe and effective alternative for thermal ablation techniques such as radiofrequency or cryoablation. Total applied current, not delivered power (watts), energy (joules), or voltage, is the parameter that most directly relates to the local voltage gradient that causes electroporation. Electroporation can be achieved with various modalities: direct current, alternating current, pulsed direct current, or any combination of these. Experimental cardiac and noncardiac studies have demonstrated tissue specificity with survival of arteries and nerves in large lesions. In addition, porcine data suggest that application inside a pulmonary vein does not lead to pulmonary vein stenosis and that the esophagus is remarkably insensitive to electroporation.

One rate-limiting factor in the uptake of these procedures is the high cost of technology used and confounded by low reimbursement by health care payers. Device-related complications such as access site complications, ischemic stroke, esophageal fistula, and phrenic nerve injury also play a role in the reluctance to offer procedures to all patients. High manufacturing costs to build challenging catheter designs as well as uncontrolled thermal treatments (i.e., cryotherapy & radiofrequency ablation) play a major role in the technological limitations in catheter ablation for AF.

Medtronic's published research entitled “Intracardiac Pulsed Field Ablation: Proof of Feasibility in a Chronic Porcine Model” noted that Pulsed field ablation (PFA) is a form of IRE that uses a train of bipolar and biphasic pulses of high voltage and short duration to create tissue injury without significant heating. This research investigated using PFA delivery to a 9-electrode circular array catheter to achieve atrial myocardial injury comparable to that achieved by duty cycled RFA, with reduced injury to nontargeted tissues. It was found that PFA technology produced targeted cardiomyocyte death, reduced EGM amplitude, and resulted in lasting atrial lesions when delivered from the multielectrode circular array catheter. The study reported that, compared to duty-cycled RF ablations, the healing characteristics of PFA lesions were devoid of a thermal signature, had an absence of lingering “sequestered” cardiomyocyte groups, had more uniform replacement fibrosis, showed significantly reduced epicardial fat inflammation, and resulted in less intralesional blood vessel remodeling, whereas both PFA and RFA deliveries were devoid of collateral damage. The study noted that further research on this new catheter ablation energy source is needed to verify reduced specific safety risks and potentially improved efficacy over existing ablation technologies.

US2017/0035499A1 to Medtronic Inc. discloses a method for ablating tissue by applying at least one pulse train of pulsed-field energy. The method includes delivering a pulse train of energy having a predetermined frequency to cardiac tissue. The pulse train of energy includes a plurality of Voltage amplitudes, at least 60 pulses, an inter-phase delay between 0 us and 5 us, an inter-pulse delay of at least 400 us, a pulse width of 1-15 us, and a voltage between 300V and 4000V. The plurality of Voltage amplitudes includes a second amplitude being higher than a first amplitude, wherein the biphasic pulses delivered at the first amplitude are delivered at a higher frequency than the biphasic pulses delivered at the second amplitude. The pulses may be short (e.g., nanosecond, microsecond, or millisecond pulse width) order to allow application of high Voltage, high current (example, 20 or more amps) without long duration of electrical current flow that results in significant tissue heating and muscle stimulation. However, such methods require specialized catheter designs at a high manufacturing cost and not flexible in application during a surgical procedure.

It would be advantageous to provide a PFA catheter design that could create larger point or spot ablation lesions to reduce the time needed to perform the procedure.

It would be advantageous to provide a PFA catheter design that could create a variety of spot ablation diameters to better integrate with differing anatomies improving medical procedure efficiency.

It would be advantageous to provide a PFA catheter design that delivers large spot ablation lesions without increasing the risks relating to thermal ablation cardiac therapies and devices.

Any discussion of prior art throughout the specification should in no way be considered as an admission that such prior art is widely known or forms part of common general knowledge in the field.

### BRIEF SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A substantial minority of patients with persistent atrial fibrillation (AF) often require pulmonary vein isolation (PVI) ablation and additional ablation at non-PV trigger sites as well as for concurrent rhythms such as atrial flutter. PVI alone is sometimes insufficient to treat persistent atrial fibrillation. A number of extra lesion sets are often needed such as mitral isthmus lines, rooflines, posterior lines, cavotricuspid isthmus (CTI) lines, posterior box isolation, left atrial appendage isolation, and ablation at punitive AF sources/rotors using a variety of AF electrogram mapping approaches. The present disclosure is designed to meet such procedural needs, wherein a single catheter may be used for both diagnostic mapping and therapeutic ablation. This reduces both workflow complexity and procedure costs.

A typical use-case scenario of the present disclosure is as follows. The small diameter fixed loop catheter used in a patient presenting with persistent atrial fibrillation (AF). The loop is inserted into the left atrium using a non-steerable 8.5F SLO or steerable 8.5F Agilis introducer. A 3D EAM of the left atrium is created by maneuvering the small diameter fixed loop catheter around the left atrium chamber, including the pulmonary veins (PV), and left atrial appendage. At the same time, voltage and activation maps are created. The small diameter fixed loop catheter is used to isolate the PVs by applying pulsed field ablation (PFA) energy creating contiguous lines comprising wide footprint lesions around the left superior pulmonary vein (LSPV), left inferior pulmonary vein (LIPV), right superior pulmonary vein (RSPV), and right inferior pulmonary vein (RIPV).

After the small diameter fixed loop catheter is used to isolate the PVs, the catheter is inserted into the PVs to check for block across ablation lines. Any gaps are detected by the small diameter fixed loop catheter and are ablated again. If AF is still persistent, the small diameter fixed loop catheter may be used to create lesions along the roof line and anterior line for posterior wall isolation. If AF continues to be persistent, the small diameter fixed loop catheter may be used to create a mitral isthmus line in the left atrium pr cavotricuspid isthmus line in the right atrium. The catheter may then remap voltage and/or activation maps to confirm the procedure is successful.

### PROBLEMS TO BE SOLVED

It would be advantageous to provide a PFA variable loop catheter capable of creating different sized spot ablation lesions without crossing of electrodes that may lead to electric surging and or arcing.

It would be advantageous to provide a large spot ablation catheter with a decreased risk of thrombus formation and stroke.

It is an object of the present disclosure to overcome or ameliorate at least one of the disadvantages of the prior art, or to provide a useful alternative.

### BRIEF DESCRIPTION OF THE DRAWINGS

The accompanying drawings, which are incorporated herein and constitute part of this specification, illustrate the presently preferred embodiments of the present disclosure, and, together with the general description given above and the detailed description given below, serve to explain the features of the present disclosure.

FIG. 1 depicts a perspective view of the first exemplary embodiment of a catheter.

FIG. 2 depicts a side elevational view, in section, of the catheter of FIG. 1.

FIG. 3 depicts a perspective view of a distal tip of the catheter of FIG. 1.

FIG. 4 depicts a front elevation view of the distal tip of FIG. 3.

FIG. 5A depicts an elevation view of a numerical solution of the electric field graph showing an exemplary PFA lesion at an 8 mm loop diameter. A dose of 1800V was prescribed using an IRE threshold of 268 V/cm do determine PFA lesion formation.

FIG. 5B depicts an elevation view of a numerical solution of the electric field graph showing an exemplary PFA lesion at a 10 mm loop diameter.

FIG. 5C depicts an elevation view of a numerical solution of the electric field graph showing an exemplary PFA lesion at a 12 mm loop diameter.

FIG. 5D depicts an elevation view of a numerical solution of the electric field graph showing an exemplary PFA lesion at a 14 mm loop diameter.

### DETAILED DESCRIPTION

In the drawings, like numerals indicate like elements throughout. Certain terminology is used herein for convenience only and is not to be taken as a limitation on the present disclosure. The terminology includes the words specifically mentioned, derivatives thereof and words of similar import. The embodiments illustrated below are not intended to be exhaustive or to limit the present disclosure to the precise form disclosed. These embodiments are chosen and described to best explain the principle of the present disclosure and its application and practical use and to enable others skilled in the art to best utilize the present disclosure.

Reference herein to “one embodiment” or “an embodiment” means that a particular feature, structure, or characteristic described in connection with the embodiment can be included in at least one embodiment of the present disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in the present disclosure, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in the present disclosure and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present disclosure.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

One preferred embodiment of the present disclosure comprises a “mid-size” 7-7.5F PFA catheter including at least four electrodes, about 2 mm in length, fixed on a small (8-10 mm) diameter fixed loop. The at least four electrodes are arranged around the small diameter fixed loop to create a large footprint focal lesion. Said lesion is volumetrically much greater in size when compared to conventional point ablation catheters. This larger lesion footprint reduces procedure time and number of lesions required by creating a wider ablation zone that overlaps for more durable contiguous lesions.

Another preferred embodiment of the present disclosure comprises a highly versatile and maneuverable catheter used to map and ablate the pulmonary veins and other intracardiac structures. The small diameter fixed loop results in closely spaced electrodes increasing mapping resolution that potentially improves diagnostic outcomes. The small diameter fixed loop may be used to create 3D EAM of the atriums, voltage maps, activation maps, and pacing to check for block.

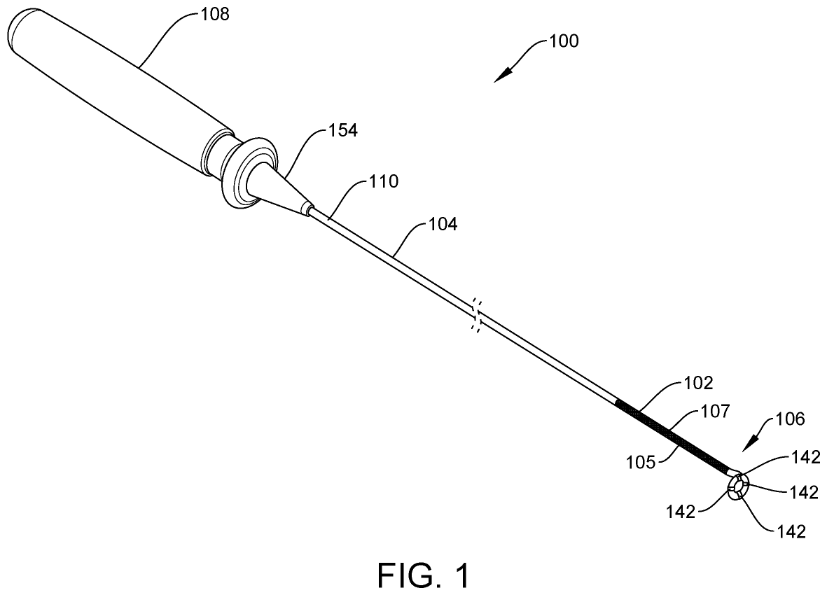

FIG. 1 depicts a perspective view of a first exemplary embodiment of the present disclosure in the form of a device or system including a small diameter fixed loop pulsed ablation catheter 100 for use in atrial fibrillation treatment. Catheter 100 can be a pulsed field ablation (PFA) catheter, such as the catheter disclosed in commonly owned U.S. Provisional Patent Application Ser. No. 63/236,750 filed on Aug. 25, 2021. Alternatively, catheter 100 can be a radiofrequency ablation (RFA) catheter and/or a combination of PFA and RFA energy deliveries.

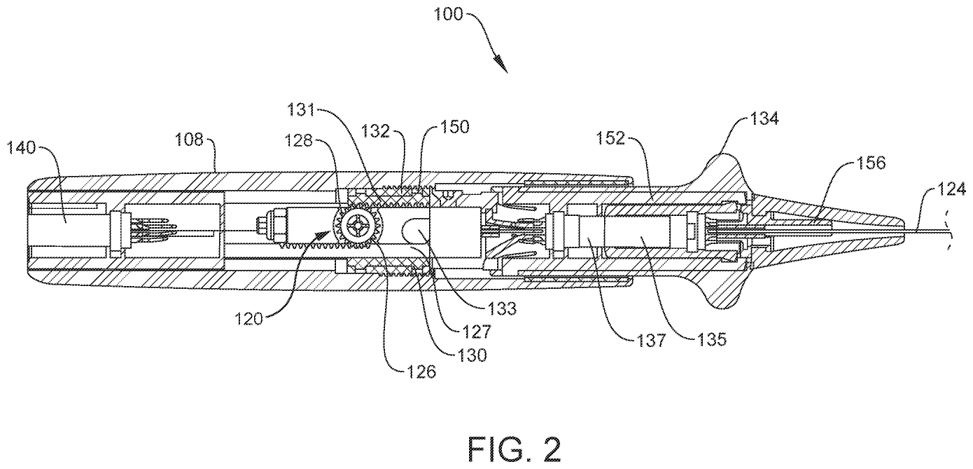

In an exemplary embodiment, the ablation catheter 100 includes a distal deflection zone 102 coupled distally to the small diameter fixed loop 106 as well as proximally to the shaft 104, and a handle 108 coupled to proximal end 110 of the shaft 104. Referring to FIG. 2, within the ablation catheter 100 is a direction imparting mechanism 120 that is configured to change the direction of the distal deflection zone 102 by manipulating a steering mechanism or knob/plunger 134 to maneuver the steerable small diameter fixed loop 106 to a treatment site, and a rotational actuator 126 configured to steer the loop 106 to better interface with human anatomy.

In an exemplary embodiment, rotational actuator 126 comprises a rack 127 and pinion 128 such that rotation of pinion 128 slides rack 127 to steer the distal deflection zone 102. Rack 127 and pinion 128 are sandwiched between two clam shells 130,132. Clam shell 130 also has a rack 131 to interface the opposing side of the pinion 128 for smoother movement of the rotational actuator 126. The rack 127 is fixed to the knob/plunger 134 that is advanced by the user to deflect the distal deflection zone 102. When the knob 134 and rack 127 are advanced, the pinion 128 advances in the opposite direction as the direction imparting mechanism interfaces with the racks 127, 131. The clam shell 130, 132 are fixed to the handle 108, but not the knob 134, and retains the pinion 128 on track with the racks 127, 131. Clam shell 130 also limits the stroke of the pinion assembly through a key feature 133. A nitinol steering tube 124 is fixed to the distal end of the rack 127 and a nitinol pullwire (not shown) that extends the longitudinal axis of steering tube 124 is attached to the pinion 128 by way of a grub screw fastened inside the key feature 133. When steering tube 124 and the pullwire are pulled by the pinion 128, causing the distal deflection zone 102 to deflect.

An exemplary steering mechanism is disclosed in U.S. patent application Ser. No. 15/550,651, filed on Aug. 11, 2017, and published as U.S. Patent Application Publication No. 2018/00365011 on Feb. 8, 2018, which is owned by the Assignee of the present disclosure and incorporated herein by reference in its entirety.

FIG. 2 also shows a handle 108 having a removable connection 150 to a direction imparting mechanism 120. In one exemplary embodiment, the removable connection 150 is a threaded connection, although those skilled in the art will recognize that other types of connections can be provided. A distal insert 152 can be removable or disposable, such that handle 108 and internals of catheter 100 re-usable. Additionally, disposable distal tip 154 is located distally of the distal insert 152. Distal tip 154 guides shaft 104 out of catheter 100 and focuses the movement of shaft 104. A delivery lumen 156 extends longitudinally through distal tip 154 such that shaft 104 extends along delivery lumen 156.

In another exemplary embodiment, the removable connection 150 is fixed in the handle 108 by way of adhesive, locking screw, key feature, or similar. The distal insert 152 is also fixed inside the knob 134. In this arrangement, connectors 135, 137 may be made redundant and electrical connections may be made directly from electrodes 142 to proximal connector 140. This may remove the chance of cross-connection and short-circuiting during high-voltage energy delivery. It also reduces the cost of the device.

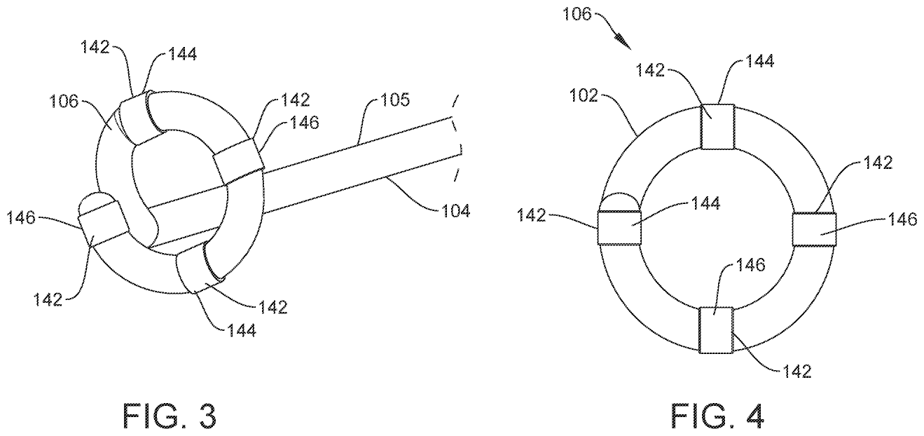

FIGS. 3 and 4 depict a small diameter fixed loop 106 with four electrodes 142, namely, alternating positive electrodes 144 and negative electrodes 146. While four electrodes 142 are shown, those skilled in the art will recognize that additional electrodes 142, in multiples of two electrodes 142, can be provided. Electrodes 142 are even spaced apart from each other; in this embodiment, each electrode 142 is spaced about 90 degrees around an arc from adjacent electrodes 142.

The small diameter fixed loop catheter 100 can be constructed from a shape memory material, such as nitinol, to allow for deformation and subsequent re-formation of the loop 106 containing electrodes 142. The small size loop 106 (whether 8 mm, 10 mm, or 12 mm in diameter) produces a continuous lesion on adjacent tissue, such as cardiac tissue, when electrical current is applied to electrodes 142. Such lesion can irreversibly electroporate tissue or irreversibly cauterize the tissue. The use of catheter 100 also reduces the risk of tissue wall perforation compared to a linear catheter, which has a higher risk of perforation.

Referring to FIGS. 1, 3, and 4, a deflection zone 105 of shaft 104 is a composite tube having a braid fiber 107 positioned at an angle to maximize flexibility and torsional resistance. The braid fiber 107 can be non-conductive or conductive braid fiber.

In an exemplary embodiment, the braid fiber 107 may be constructed, partially or entirely, of a non-metallic material to prevent or limit cross-connecting or short-circuiting of the catheter 100 during energy delivery. The braid fiber 107 can be constructed of a reinforced nylon, polyurethane, PEEK, or Kevlar (liquid crystal polymer) material.

The catheter 100 of the present disclosure delivers voltages greater than 300 volts. The voltage passes through the catheter shaft 104 through small, insulated wires (not shown). The wires pass parallel to the nitinol shape imparting mechanism and terminate at the plurality of electrodes 142 on loop 106. Each wire corresponds to a single electrode 142. In some embodiments, the electrodes 142 are arranged in electrode pairs 144, 146, otherwise known as channels. Each electrode pair 144, 146 is synchronized to deliver either bipolar monophasic waveforms or bipolar biphasic waveforms. The synchronized pulse delivery works by one electrode 144, 146 in each channel emitting a voltage and a current. That voltage and current then passes through the resistive heart tissue and exits through the other electrode 146, 144 of the pair. Because high voltages greater than 300 volts are expected, the construction of loop 106 under the electrodes 142 is made of an insulative material. In an exemplary embodiment, the shaft 104 is constructed of a silicone polymer.

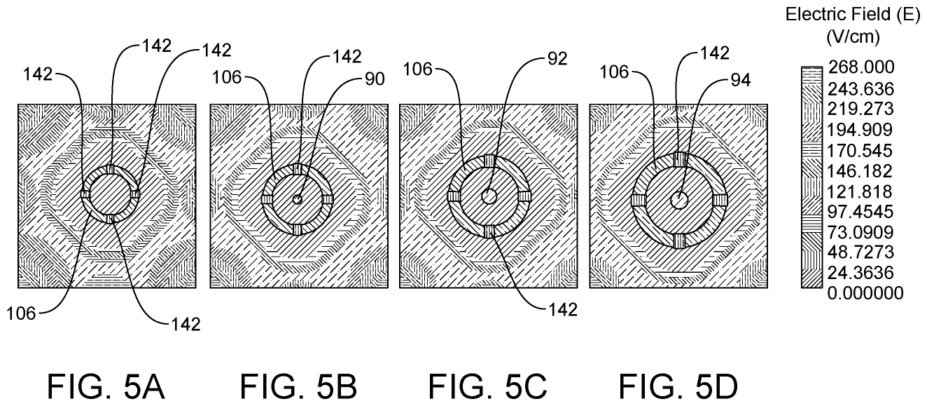

FIGS. 5A-5D each depicts an elevation view of a numerical solution of the electric field graph showing an exemplary PFA lesion at 8 mm, 10 mm, 12 mm, and 14 mm loop diameters, respectively. A dose of 1800V was prescribed using an IRE threshold of 268 V/cm do determine PFA lesion formation. Loop 106 is shown in each Figure and electrodes 142 are shown in FIG. 5A for clarity. The light spots 90, 92, 94 in the centers of FIGS. 5B, 5C, 5D, respectively, show tissue that is not ablated due to the larger diameter of the tip relative to the tip shown in FIG. 5A with the 8 mm diameter.

Additionally, further embodiments may include an additional feature (not shown) wherein the shaft 104 includes metallic or metallized braid up to the start of the deflection zone of the shaft 104, and extending beyond the start of the deflection zone, the braid is constructed of a non-metallic braid up to the loop 106 at the distal end of shaft 104. Preferably, the region underneath or close to the electrodes 142 can be non-metallic braid.

### Calculation of Electric Fields

To determine the electric field volume created by the small diameter fixed loop catheter, an in-silico model was designed to simulate electric fields using electrostatic finite element analysis. The model geometry comprises a blood domain, heart tissue, and the small diameter fixed loop catheter.

Electrostatic finite element analysis uses Gauss's law and the more mathematically complex Maxwell's equations. These equations solve for charge and voltage distribution across a medium. To solve such complex numerical calculations, Electromagnetic Works (EMS) 2018 multi-core iterative electrostatics solver is used. This finite element method (FEM) software computes the following Maxwell equations:

$$∇ \times E = 0 \qquad ( 1 )$$

$$∇ \cdot D = ρ \qquad ( 2 )$$

where E and D are the electric and displacement fields, respectively. The ∇×symbol denotes the divergence operator and ∇·symbol denotes the curl operator, where ρ is the charge density. To solve equations 1 & 2 a constitutive relationship is applied to form:

$$D = ε E \qquad ( 3 )$$

where ϵ is the permittivity of the material. The equation is further simplified by introducing the electric potential φ that forms:

$$E = - ∇ φ \qquad ( 4 )$$

The famous Poisson's equation (4) can then be obtained from equations 1 & 2. Finally, the electrostatic analysis executes the following Poisson's equation:

$$∇ \cdot ( ε ∇ φ ) = ρ \qquad ( 5 )$$

The Poisson's equation (5) can be solved for a given model of a PFA catheter assembly and its surrounding medium by FEM using EMS or related software. The model imposes input boundary conditions such as the amplitude of φ being constant on the electrode surface and the E vector being parallel to insulative surfaces such as the insulated catheter shaft. Without the aid of FEM computer programs, computing the above problem is quite challenging. Physical properties and input boundary conditions are specified below.

Tissue conductivities are challenging to predict as they are a function of local electric field intensity and temperature. For myocardium, the temperature-dependent piecewise thermal conductivity function grows linearly 0.12° C.⁻¹ up to 100° C. and then is kept constant. Alternatively, heart tissue's electrical conductivity features an exponential growth of 1.5° C.⁻¹ between 0 and 100° C. Although temperatures beyond 100° C. are not likely in PFA, heart tissue does experience a linear decay of 4 orders of magnitude for 5° C. that models the tissue desiccation at 100° C., and then remains constant. To compound the problem further, experimental values for tissue conductivities may also increase due to continued pore formation where the cytoplasm opens previously unavailable intracellular current pathways. This phenomenon likely relates to the waveform type, including monopolar or bipolar pulse deliveries and the number of pulses. For example, Tekle et al. reported that bipolar square waves permeabilized cell membranes better than unipolar square waves, and Garcia et al. illustrated the importance of higher pulse numbers leading to larger cell kill counts. Such tissue conductivity and waveform effects can be analyzed experimentally and defined empirically, which can be combined with heating effects to form constituents of dynamic electrical conductivities.

To execute the dynamic behavior of heart muscle during PFA would require complex thermal-fluid modelling or difficult experimental measurements, then extensive ex vivo tissue characterization data. Also, the overall goal of this work is to inform a PFA system design and provide insight into PFA lesion effects, potentially limiting the need for costly in vivo testing. As such, this current report uses readily available ex vivo electroporation data obtained from the kidneys.

Although the heart and the kidneys are quite distinct by way of function, looking deeper reveals tissue properties quite similar by comparison. Take, for example, tissue thermal conductivity, the heart muscle and kidney tissue have thermal conductivities of 0.56 and 0.53 Wm⁻1° C.⁻¹, respectively, (ITIS database, Zurich, SWITZERLAND). Therefore, the electrical conductivity percent increases, as a function of temperature, that was obtained in the kidney during ex vivo electroporation, would likely provide a conservative yet relatively accurate alternative to heart muscle dynamic conductivities during PFA. In this respect, a tissue conductivity at 45 kHz was selected and an approximated 18% increase in conductivity, as might be expected when delivering >20 pulses, was added. These tissue properties would result from a waveform comprising two 15 μs biphasic pulses and two 5 μs inter-pulse delays with added (0.0347 Sm⁻¹) conductivity. This translates to about 150 kHz or very fast 3.325 us biphasic pulses with no inter-pulse delays, which may be needed during high-voltage delivery, as disclosed in commonly owned U.S. Provisional Patent Application Ser. No. 63/236,750 filed on Aug. 25, 2021. As for the tissue permittivity, this was estimated based on typical values at the predetermined tissue conductivities. Similarly, the blood properties reflect the material properties occurring during the faster direct current (DC) PFA waveforms of 5us pulse durations. However, the blood was neglected during electric field assessment as the electric fields in the blood are of little relevance to lesion assessment.

| Material | ε (Fm⁻¹) | σ (Sm⁻¹) |
|---|---|---|
| Blood | 5120 | 0.703 |
| Heart Muscle | 7330 | 0.228 |
| Pure Gold | — | 41,000,000 |
| PTFE | — | 0 |

Boundary conditions also reflect Neal et al. work where the anode is energized with a prescribed voltage, and the cathode is set to ground. In this current work, one electrode is energized while the other electrode is set to the ground. This boundary condition creates an electric field as the flow of current passes from one electrode and travels through the surrounding anatomical space and enters the grounded electrode. This study is a steady-state calculation meaning pulse width and number of pulses is not possible with this current analysis. Instead, the increased cell kill count due to waveform type is captured in the 18% increase to the material property's electrical conductivity and the resulting permittivity. As a result, the electrical field simulated between the two electrodes depicts the lesion depth due to the assumed heart tissue's dynamic conductivity taken from ex vivo experimentation with kidneys. Further, this is made possible by a known IRE threshold of heart tissue of 268 V/cm. This also means that blood flow is not calculated for. Electric field profiles will not be affected by the lack of blood flow, which is particularly useful in thermal fluid modelling where passive cooling of electrodes is apparent as disclosed in commonly owned U.S. Provisional Patent Application Ser. No. 63/236,750 filed on Aug. 25, 2021.

In this study, cardiac PFA lesions were identified using an IRE threshold of 268 V/cm. This threshold was first captured during unipolar monophasic PFA, which is indeed a markedly different waveform than is being employed by the clinical Medtronic and Farapulse systems used for validation purposes. Alternatively, a threshold of 400 V/cm is generally proposed. The selection of 400 V/cm as a threshold in previously published studies is based on in vitro experimentation using rat myoblasts and defines an approximate threshold of 375 V/cm. The IRE threshold of rat myoblasts determined by Kaminska et al. is an incomplete data set suggesting an IRE threshold greater than 300 V/cm and less than or equal to 375 V/cm. Therefore, the current evidence indicates an average IRE threshold of myocardioytes to be 322±54 V/cm rather than 400 V/cm.

Histopathology shows transitions from reversibly to irreversibly electroporated cells occur continuously throughout an electroporated lesion. Therefore, irreversible electroporation thresholds cannot be regarded as discrete values, nor can they be based on a single in vitro study. Instead, IRE threshold estimation is generally made by overlapping histological results with the computational model's electric field distribution. The computational model proposed herein revealed an IRE threshold of 268 V/cm reported by Wittkampf et al. using a generalizable PFA waveform and tissue properties when overlapped with histological results reported by Medtronic and Farapulse. In a recent study, Calouri et al. used their own PFA waveform combined with RFA tissue properties that are more conductive than those used in this current computational model but reported the histological results remained superior to those predicted computationally. Although the authors contribute this to the effect of multiple pulses not accounted for in their model, it may also be due to the higher IRE threshold of 400 V/cm used to interpret the results as this would greatly reduce the IRE isotherm. A lower IRE threshold of 322±54 V/cm would undoubtingly improve the computational prediction of the PFA boundary.

Finally, the waveform and pulse parameters used during in vitro experimentation on rat myoblasts were also different from those currently employed by Medtronic and Farapulse. The waveform used was a monophasic square wave, whereas Medtronic and Farapulse currently use a biphasic square wave. Inasmuch, a biphasic square wave induces a more significant cellular response than a monophasic square wave. Additionally, a high-tilt exponential pulse, such as the one used by Wittkampf et al., may produce less injury than rectangular pulses. Therefore, there is reason to believe that cardiac myocytes may elicit an even lower IRE threshold than currently proposed by others. For example, Oliveira et. al shows rat ventricular myocytes experiencing IRE at electric fields strengths as little as 50 V/cm. A typical IRE threshold induced by PFA is suspected to be greater than this value, but less than 350 V/cm.

It will be further understood that various changes in the details, materials, and arrangements of the parts that have been described and illustrated in order to explain the nature of this present disclosure may be made by those skilled in the art without departing from the scope of the invention as defined by the following claims.

1. A mini-loop cardiac ablation catheter comprising:
a) a shaft having a proximal end, a distal end, and at least one loop extending from the distal end;
b) a handle coupled to the proximal end of the shaft, the handle having a steering mechanism; and
c) a plurality of electrodes positioned on the loop electrically coupled to at least one electrical connector in the handle, the at least one electrical connector being configured to electrically coupled with an electrical ablation energy source to power the plurality of electrodes.

2. The catheter according to claim 1, wherein the loop is steerable.

3. The catheter according to claim 1, wherein the plurality of electrodes are spaced at predetermined intervals along the loop.

4. The catheter according to claim 3, wherein the predetermined intervals are equidistant.

5. The catheter according to claim 1, wherein the plurality of electrodes comprises four electrodes.

6. The catheter according to claim 1, wherein the loop has a diameter no larger than 12 mm.

7. The catheter according to claim 1, wherein the loop is between 8 mm and 12 mm in diameter.

8. The catheter according to claim 1, wherein the loop comprises a non-conductive material.

9. The catheter according to claim 1, wherein the steering mechanism comprises a rack and pinion.

10. The catheter according to claim 9, wherein the pinion is operated by a knob.

11. The catheter according to claim 1, wherein the distal end of the shaft is braided.

12. The catheter according to claim 11, wherein the braid is constructed of a non-electrically conductive material.
